(19) United States
(12) Patent Application Publication
(10) Pub. No.: US 2005/0283047 A1
(43) Pub. Date: Dec. 22, 2005

(54) Endoscopic system

(75) Inventors: Koichi Tashiro, Sagamihara-shi (JP); Takeaki Nakamura, Tokyo (JP); Akinobu Uchikubo, Iruma-shi (JP)

Correspondence Address: SCULLY SCOTT MURPHY & PRESSER, PC, 400 GARDEN CITY PLAZA, SUITE 300, GARDEN CITY, NY, 11530, US

(73) Assignee: OLYMPUS CORPORATION, TOKYO (JP)

(21) Appl. No.: 11/157,447

(22) Filed: Jun. 21, 2005

(30) Foreign Application Priority Data
Jun. 21, 2004 (JP) 2004-182133

(51) Int. Cl. A61B 1/04

(52) U.S. Cl. 600/118; 600/109

(57) ABSTRACT

An endoscopic system including a system controller for controlling at least an endoscope and an AV controller for controlling one or more audio-visual devices, the AV controller being capable of communicating with the system controller, wherein the system controller controls the one or more audio-visual devices. For this purpose, the system controller exchanges data for use in activating the one or more audio-visual devices with the AV controller. In this data communication, the system controller can transmit a setting list to the AV controller, for example, or a link may be established between the system controller and the AV controller.

### CROSS-REFERENCE TO RELATED APPLICATION

This application is based upon and claims the benefit of priority from prior Japanese Patent Application No. 2004-182133, filed Jun. 21, 2004, the entire contents of which is incorporated herein by reference.

### BACKGROUND OF THE INVENTION

1. Field of the Invention

The present invention relates to an endoscopic system having an endoscope.

2. Description of the Related Art

In recent years, a wide variety of medical devices have been developed along with an advance of medical technologies and their features tend to be expanded. A diverse range of medical devices are available such as an electric cautery, and a laser surgical knife. These medical devices are used singly in some cases or in combination as a medical system composed of a plurality of medical devices in other cases.

This type of medical system includes an endoscopic system having an endoscope and medical devices other than the endoscope as suggested in Laid-Open Japanese Patent Publication Nos. 2003-76786 and 2003-70748, for example.

In the foregoing conventional endoscopic system, the medical devices have the same communication interface or communication protocol. Thereupon, the endoscopic system integrally controls the various medical devices by using a system controller via a communication section.

The foregoing conventional endoscopic system is placed and used in an operating room. On the other hand, audio-visual (AV) devices are placed in the operating room. These AV devices include, for example, a room light, a room camera, display devices such as a liquid crystal display (LCD) and a plasma display panel (PDP), a compact disc (CD)®, a digital versatile disc (DVD), a video tape recorder (VTR), and a server for storing reference images such as ultrasound images.

There are numerous types of AV devices and their communication interfaces or communication protocols are different from those for use in controlling medical devices. Therefore, it has been difficult for the conventional endoscopic system to control these AV devices. Thereupon, a dedicated AV controller has been used to control the AV devices.

When the foregoing AV controller is used, the conventional endoscopic system controls desired medical devices by operating an operating panel of the system controller, while controlling desired AV devices by operating a touch panel for the AV controller.

Accordingly, a surgeon who uses the conventional endoscopic system needs to walk around between the two panels to operate desired medical devices and AV devices, which leads to complicated operations.

### BRIEF SUMMARY OF THE INVENTION

The present invention relates to an endoscopic system comprising a system controller for controlling at least an endoscope and an AV controller for controlling one or more audio-visual devices. The system controller communicates with the AV controller. In the endoscopic system, the system controller has a function of transmitting or receiving data for use in activating the audio-visual devices to or from the AV controller by communicating therewith.

Therefore, it is possible to control the audio-visual devices via the system controller and the AV controller from the side of the endoscope. Thus, the operator can operate both of the medical devices including the endoscope and the audio-visual devices only by using an operating panel for the medical devices including the endoscope, which frees the operator from the inconvenience of using a plurality of operating panels.

For example, the endoscopic system can be configured in such a way that the system controller transmits data for use in activating the one or more audio-visual devices to the AV controller and the AV controller checks the data received from the system controller.

This enhances reliability since the data from the system controller is checked on the side of the AV controller. Moreover, the control on the side of the system controller can be readily brought into agreement with the control on the side of the AV controller.

For example, the system controller can request the AV controller to transmit data and make settings of the one or more audio-visual devices on the basis of the data acquired from the AV controller.

This allows the system controller to make the settings of the one or more audio-visual devices directly, thereby enabling more accurate settings.

Moreover, the system controller and the AV controller can be connected to different recording mediums to make them record data on the operation of the one or more audio-visual devices. In this instance, the system controller transmits the data retrieved from the recording medium on the side of the system controller to the AV controller and the AV controller sets the corresponding data in the recording medium on the side of the AV controller.

This allows each controller to hold data on the operation of the audio-visual devices in the recording medium independently, thereby increasing independence of each controller and facilitating a system construction.

Furthermore, the system controller may set up a link with the AV controller and make settings from an input device of the system controller. This enables more accurate setting operations.

### BRIEF DESCRIPTION OF THE SEVERAL VIEWS OF THE DRAWINGS

These and other features, aspects, and advantages of the apparatus and methods of the present invention will become better understood with regard to the following description, appended claims, and accompanying drawings where:

FIG. 1 is a configuration diagram of an endoscopic system according to a first embodiment;

FIG. 2 is a block diagram showing a connecting relation between the endoscopic system and an AV device system;

FIG. 3 is an explanatory diagram showing an automatic setting of AV devices through communications between a system controller and an AV controller;

FIG. 4 is a conceptual diagram showing system list data with a surgeon's name used as a keyword;

FIG. 5 is a conceptual diagram showing system list data with the name of a surgical site used as a keyword;

FIG. 6 is a conceptual diagram showing system list data with the name of a department of a hospital used as a keyword;

FIG. 7 is a block diagram showing a connecting relation between an endoscopic system according to a second embodiment and an AV device system; and

FIG. 8 is a conceptual diagram showing AV list data.

### DETAILED DESCRIPTION OF THE PREFERRED EMBODIMENTS

Preferred embodiments of the invention are described below with reference to the accompanying drawings.

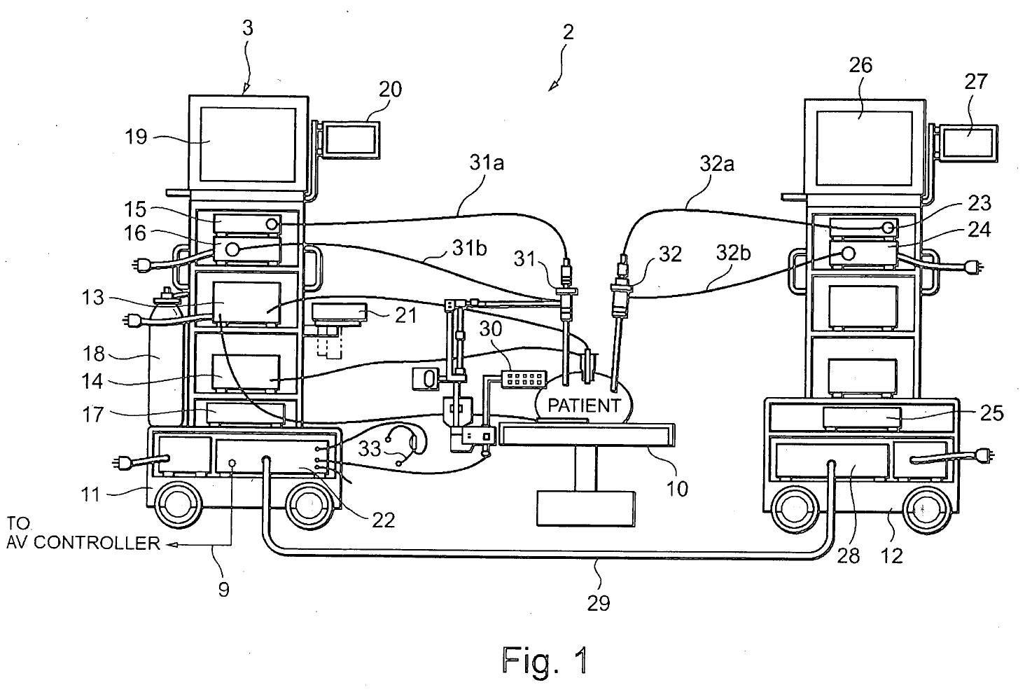

First, the configuration of an endoscopic system 3 placed in an operating room 2 will be described hereinafter by reference to FIG. 1.

As shown in FIG. 1, a patient's bed 10 on which a patient 48 is laying and the endoscopic system 3 are placed in the operating room 2. The endoscopic system 3 has a first cart 11 and a second cart 12.

The first cart 11 is loaded with medical devices as controlled devices such as, for example, an electric cautery 13, an insufflator 14, an endoscopic camera 15, a light source 16, a video tape recorder (VTR) 17, and other devices and a gas cylinder 18 filled with carbon dioxide. The endoscopic camera 15 is connected to a first endoscope 31 via a camera cable 31a. The light source 16 is connected to the first endoscope 31 via a light guide cable 31b.

Moreover, the first cart 11 is loaded with a display device 19, a first integrated display panel (hereinafter, simply referred to as display panel) 20, an operating panel 21, and the like. The display device 19 is, for example, a TV monitor for displaying endoscopic images or the like.

The display panel 20 is a display member capable of selectively displaying various data during surgery. The operating panel 21 comprises a display section such as, for example, a liquid crystal display, and a touch sensor integrally arranged on the display section, for example. The operating device 21 is an integrated operating device, which is operated by a nurse or the like in a nonsterilized area.

Furthermore, the first cart 11 is loaded with a system controller 22, which is a controller. The system controller 22 is connected to the electric cautery 13, the insufflator 14, the endoscopic camera 15, the light source 16, and the VTR stated above via communication lines (not shown). The system controller 22 can be connected to a headset-type microphone 33. Thereby, the system controller 22 can recognize voice input from the microphone 33 and can control devices according to a surgeon's voice.

On the other hand, the second cart 12 is loaded with an endoscopic camera 23, a light source 24, an image processing device 25, a display device 26, and a second display panel 27, which are controlled devices.

The endoscopic camera 23 is connected to a second endoscope 32 via a camera cable 32a. The light source 24 is connected to the second endoscope 32 via a light guide cable 32b.

The display device 26 displays endoscopic images picked up by the endoscopic camera 23. The second display panel 27 can selectively display various data during surgery.

The endoscopic camera 23, the light source 24, and the image processing device 25 are connected to a repeater unit 28 mounted on the second cart 12 via communication lines (not shown). The repeater unit 28 is connected to the system controller 22 mounted on the first cart 11 via a trunk cable 29.

The system controller 22 can integrally control the camera 23, the light source 24, and the image processing device 25 mounted on the second cart 12 and the electric cautery 13, the insufflator 14, the camera 15, the light source 16, and the VTR 17 mounted on the first cart 11. If the system controller 22 is communicating with these devices, the system controller 22 can display the setting conditions of the connected devices and the setting screens of control switches on the liquid crystal display of the operating panel 21. Moreover, the system controller 22 can enter an operation such as a change of a setting value through an operation on the touch sensor in a given area in response to a touch on the setting screen of a desired control switch.

A remote controller 30 is a second integrated operating device operated by a surgeon or the like in the sterilized area. It can control other devices, for which the communication is established, via the system controller 22.

The system controller 22 is connected to a patient monitor system 4 via a cable 9. As described later, the system controller 22 can analyze biological information acquired from the patient monitor system 4 and can display the analysis result on a required display device.

Moreover, the system controller 22 is equipped with an infrared communication port (not shown), which is a communication member. The infrared communication port is provided in a position where it is readily irradiated with infrared rays such as, for example, in the vicinity of the display device 19 and is connected to the system controller 22 via a cable.

The endoscopic system 3 is placed in the operating room 2 as stated above. In the operating room 2, however, a room light, a room camera, display devices (LCD and PDP), a CD®, a DVD, a VTR, a server for storing reference images such as ultrasound images, and other AV devices are arranged as described later.

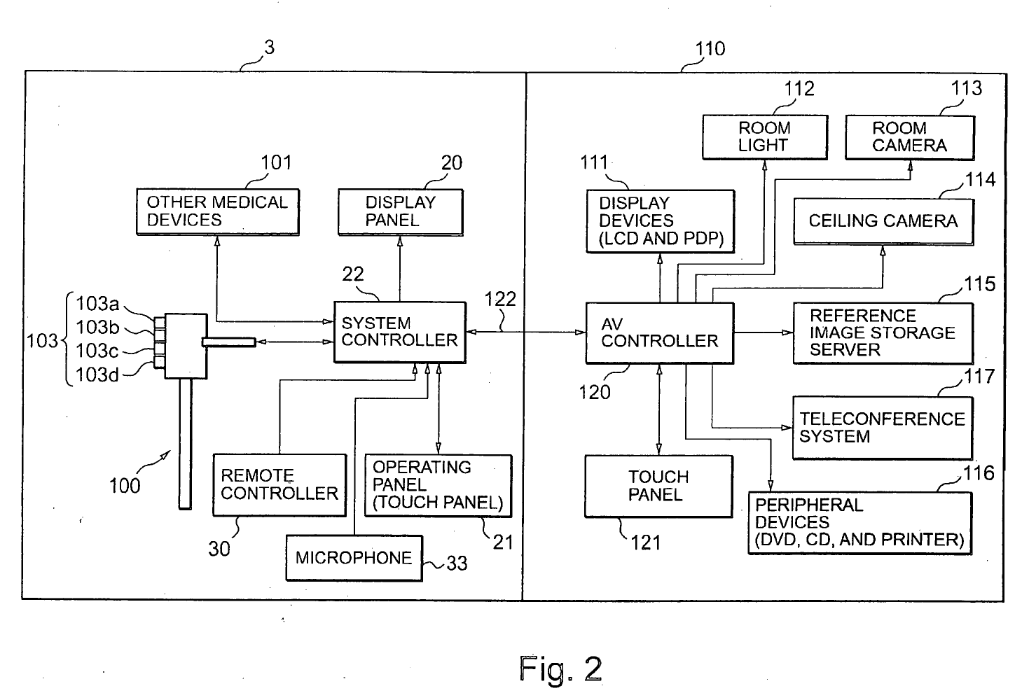

These AV devices are connected to the AV controller 120 (See FIG. 2) so as to be controlled.

The system controller 22 is connected to the AV controller 120, whereby it can communicate with the AV controller.

As shown in FIG. 2, in the endoscopic system 3, an endoscope 100 and medical devices other than the endoscope are connected to the system controller 22. The endoscope 100 corresponds to the first endoscope 31 and the second endoscope 32. Other medical devices 101 correspond to the electric cautery 13, the insufflator 14, the endoscopic camera 15, and the light source 16 described by referring to FIG. 1, for example.

Moreover, as stated above, the system controller 22 is connected to the display panel 20 and the operating panel 21 and integrally controls the endoscope 100 and other medical devices 101. The endoscope 100 and other medical devices 101 are controlled by a surgeon's operations through the remote controller 30 or the microphone 33 as stated above.

The endoscope 100 is provided with remote switches 103a to 103d in the operating section. By operating the remote switches 103a to 103d, switch signals are entered into the system controller 22, whereby other medical devices 101 (for example, the endoscopic camera 15) can be operated by remote control. With a setting operation of allocating control commands to the remote switches 103a to 103d, desired operations can be specified and controlled for desired medical devices.

Moreover, the system controller 22 has an automatic setting function of automatically making desired settings for desired medical devices among the other medical devices 101 by entering information, such as a keyword. The keyword can be a surgeon's name or a surgical procedure.

More specifically, desired setting data is previously stored in a memory, which is not shown, for keywords (a surgeon's name, a surgical procedure, or the like). Then, a surgeon or the like enters a keyword such as a surgeon's name or a surgical procedure. Thereupon, the system controller 22 reads the setting data from the memory by using the automatic setting function, selects desired medical devices specified by the setting data, and sets desired control commands and setting values specified by the setting data for the selected medical devices. Thus, the automatic setting function is for use in automatically setting desired control commands and desired setting values for desired medical devices among the medical devices 101.

An AV device system 110 is also placed in the operating room 2. The AV device system 110 includes display devices 111 such as an LCD, PDP, and the like, a room light 112, a room camera 113, a ceiling camera 114, a reference image storage server 115, peripheral devices 116 such as a DVD, CD(R), and a printer, and a teleconference system 117.

These AV devices 111 to 117 are connected to the AV controller 120 so as to be controlled. The AV controller 120 is connected to a touch panel 121. With the operation of the touch panel 121, the AV controller 120 specifies and controls a desired device among the AV devices 111 to 117.

The AV controller 120 is connected to the system controller 22 via a communication cable 122, whereby the AV controller 120 can communicate with the system controller 22.

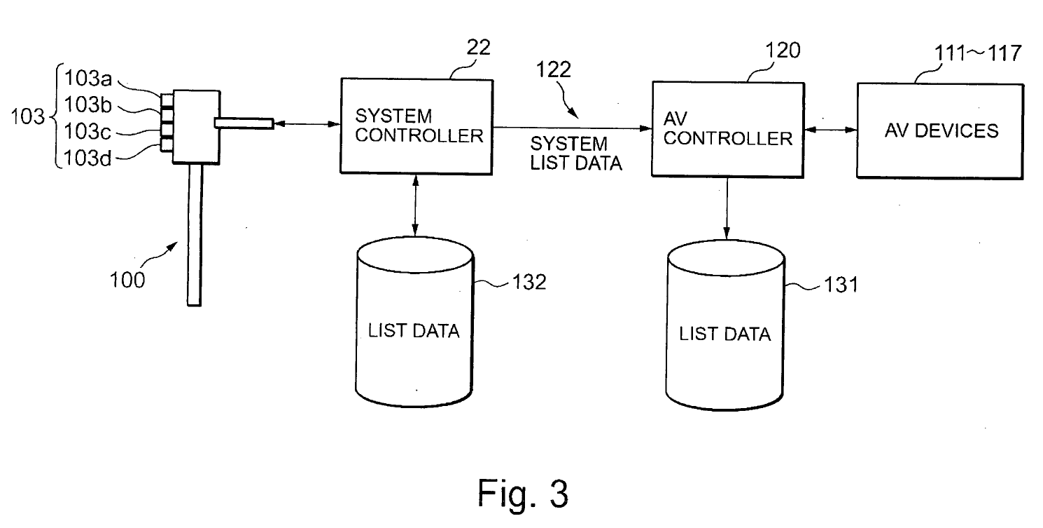

In this embodiment, automatic settings for desired devices are performed through the communication between the AV controller 120 and the system controller 22. This automatic setting operation will be described with reference to FIGS. 3-6.

As shown in FIG. 3, the AV controller 120 has an AV memory 131 for storing AV list data including connected AV devices, control commands, and setting values. On the other hand, the system controller 22 has a system memory 132 for storing system list data including control commands for AV devices and setting values with a keyword, such as a surgeon's name or a surgical procedure.

The system controller 22 reads the system list data from the system memory 132 corresponding to an entered keyword and transmits the corresponding system list data to the AV controller 120.

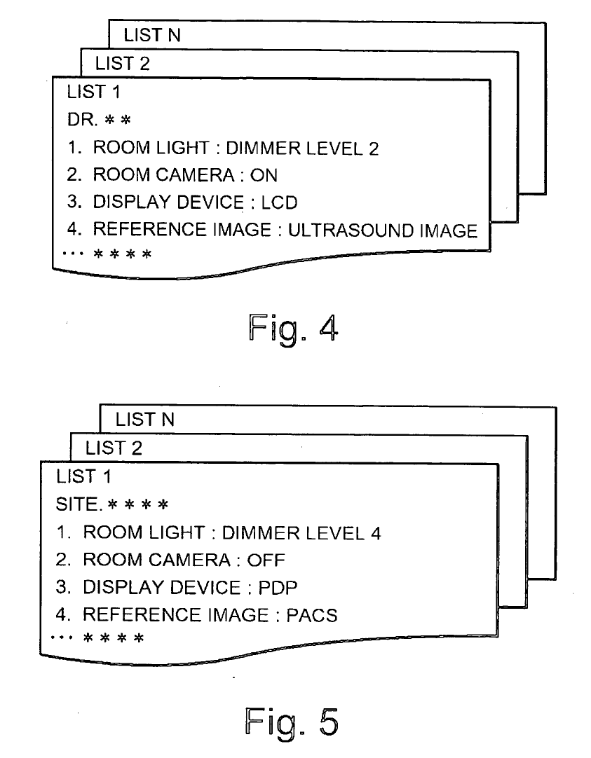

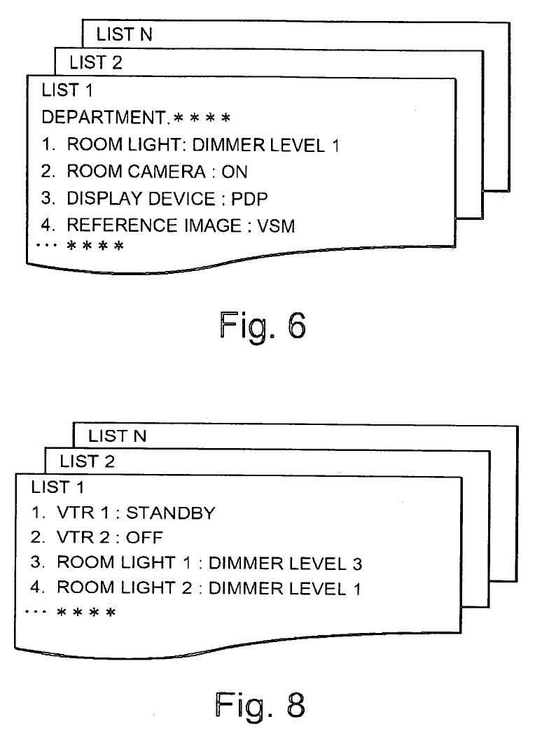

In this regard, for example, the system list data is as shown in FIG. 4, 5, or 6. The list data shown in FIG. 4 is arranged using a surgeon's name as a keyword. The list data shown in FIG. 5 is arranged using the name of a surgical site as a keyword. The list data shown in FIG. 6 is arranged using a department of a hospital as a keyword.

The AV controller 120 compares the system list data received from the system controller 22 with the AV list data read from the AV memory 131. The AV controller 120 having compared the list data makes settings for AV devices according to the system list data. More specifically, the AV controller 120 selects devices out of the connected AV devices and sets desired control commands and setting values to the selected devices. Thereby, the endoscopic system 3 can make automatic settings of the desired AV devices.

After the automatic setting of the AV devices, the desired control commands for desired AV devices among the AV devices 111 to 117 can be allocated to the remote switches 103a to 103d of the endoscope 100, in the same manner as for the allocation of the control commands for the aforementioned other medical devices 101.

The AV controller 120 allocates control commands of the desired AV devices such as, for example, those of turning on or off or dimming the room light 112 to the remote switches 103a to 103d of the endoscope 100 through operations on the touch panel 121.

Thereby, if an operator operates the remote switches 103a to 103d, the endoscopic system 3 transmits switch signals to the AV controller 120 via the system controller 22, whereby the allocated control commands are executed. In this regard, the control commands may be allocated to function keys (not shown) of the remote controller 30.

The endoscopic system 3 is configured as described with reference to FIG. 1 and the AV devices 111 to 117 are connected to the system controller 22 as described with reference to FIG. 2 for use in an endoscopic surgery.

First, a nurse or the like makes the automatic setting of medical devices before surgery. Furthermore, the nurse or the like makes the automatic setting of desired devices among the AV devices 111 to 117 as stated above.

For example, the nurse or the like enters a keyword by controlling the operating panel 21 and selects and determines the list data of Dr. **. Thereupon, the system controller 22 reads the list data with Dr. ** used as a keyword from the system memory 132 and transmits it to the AV controller 120.

The AV controller 120 reads the list data from the AV memory 131 by using Dr. ** as a keyword and compares the readout list data with the list data from the system controller 22.

The AV controller 120 having compared the list data selects devices to be set out of the connected AV devices 111 to 117 and sets desired control commands and setting values to the selected devices. Thus, in the endoscopic system 3, automatic setting of the desired AV devices is performed.

Moreover, after the automatic setting of the AV devices, the surgeon or nurse allocates desired control commands of the desired AV devices to the remote switches 103a to 103d of the endoscope 31, 32 (100).

The surgeon or nurse selects and determines control commands of desired AV devices to the remote switches 103a to 103d of the endoscope 31, 32 (100) by controlling the touch panel 121. Thereupon, the AV controller 120 allocates the selected control commands or the like to the remote switches 103a to 103d of the endoscope 31, 32 (100).

Thereby, the endoscopic system 3 transmits switch signals to the AV controller 120 via the system controller 22 through the operation of the remote switches 103a to 103d of the endoscope 31, 32 (100), whereby the allocated control commands become executable.

As stated hereinabove, the preparation for a surgical procedure is completed and then an endoscopic surgery is started.

The surgeon operates desired medical devices while viewing the display devices 19 and 26 or the display panel 20 to perform procedures for the affected area of a patient.

A subject image picked up by the endoscope 31, 32 (100) is signal-processed in a signal processing section of the endoscopic camera 15, 23 and is output to the display device 19, 26 via the system controller 22. The endoscopic image is then displayed on the display device 19, 26.

Furthermore, the display panel 20 displays operating states and setting states of other medical devices 101 such as the electric cautery 13, the insufflator 14, the endoscopic camera 15, and the light source 16. The surgeon operates the operating panel 21, the remote controller 30, or the microphone 33 to issue operating instructions of setting values or the like for the other medical devices 101 for activation.

Moreover, the surgeon operates the operating panel 21, the remote controller 30, or the microphone 33 to issue an operating instruction for a desired device among the AV devices 111 to 117.

For example, the surgeon says into the microphone 33, “Start the reference image.” Thereupon, the system controller 22 transmits a voice signal from the microphone 33 to the AV controller 120. The AV controller 120 controls and drives the reference image storage server 115 to display an ultrasound image on the display device 111.

Furthermore, for example, the surgeon dims the room light 112 if he or she wants to concentrate attention only on the endoscopic image. It is assumed here that a dimmer level 2 of the room light 112 is allocated to the remote switch 103a among the remote switches 103a to 103d of the endoscope 31, 32 (100), for example.

In this instance, the surgeon controls the remote switch 103b of the endoscope 31, 32 (100). Thereupon, a switch signal of the remote switch 103b is transmitted to the AV controller 120 via the system controller 22. The AV controller 120 receives the switch signal of the remote switch 103b and dims the room light 112 from a previous dimmer level to the dimmer level 2.

Thus, the endoscopic system 3 of the first embodiment can control the AV devices 111 to 117 from the AV controller 120 via the system controller 22.

As a result, the endoscopic system 3 of the first embodiment can operate the AV devices with a simple configuration.

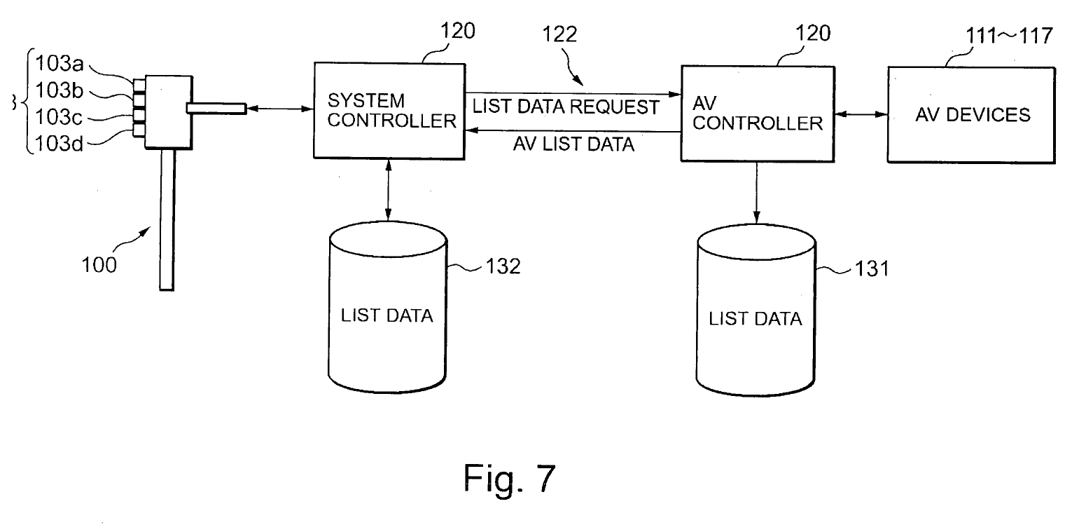

FIG. 7 and FIG. 8 relate to a second embodiment of the present invention. Referring to FIG. 7, there is shown a block diagram showing a connecting relation between an endoscopic system according to the second embodiment and an AV device system. Referring to FIG. 8, there is shown a conceptual diagram showing AV list data.

In the second embodiment, the system controller 22 requires list data and a link is provided by viewing and setting returned AV list data on the operating panel 21. Other parts are the same as those of the first embodiment. Therefore, their description is omitted here and the same reference numerals are used for the same parts in the following description.

More specifically, as shown in FIG. 7, in the endoscopic system 3 of the second embodiment, a system controller 22 requests an AV controller 120 to transmit AV list data according to a keyword that has been entered.

The AV controller 120 transmits AV list data read from the aforementioned AV memory 131 according to the keyword to the system controller 22 on the basis of the list request from the system controller 22.

The system controller 22 receives the AV list data from the AV controller 120, whereby the AV list data can be viewed using the operating panel 21. The AV list data is data as shown in FIG. 8, for example.

Viewing the AV list data using the operating panel 21, the surgeon or nurse can select a desired AV device by entering a keyword into the system controller 22 and set a desired control command and a setting value to the selected device.

Thereby, the endoscopic system 3 can provide a link between the system controller 22 and the AV controller 120 and can make an automatic setting of the desired AV device.

Accordingly, the endoscopic system 3 of the second embodiment can achieve the same effect as in the first embodiment and further can provide the link between the system controller 22 and the AV controller 120, thereby enabling a more accurate setting.

Other embodiments or the like composed of the above embodiments partially combined are also within the scope and spirit of the present invention.

While there has been shown and described what are considered to be preferred embodiments of the invention, it will, of course, be understood that various modifications and changes in form or detail could readily be made without departing from the spirit of the invention. It is therefore intended that the invention not be limited to the exact forms described and illustrated, but constructed to cover all modifications that may fall within the scope of the appended claims.

1. An endoscopic system comprising:
a system controller for controlling at least an endoscope; and
an AV controller for controlling one or more audio-visual devices, the AV controller being capable of communicating with the system controller, wherein
the system controller has a function of transmitting and/or receiving data to and/or from the AV controller for use in activating the one or more audio-visual devices by communicating therewith.

2. The endoscopic system according to claim 1, wherein:
the system controller transmits data for use in activating the one or more audio-visual devices to the AV controller; and
the AV controller checks the data received from the system controller.

3. The endoscopic system according to claim 1, wherein:
the system controller requests the AV controller to transmit data and makes settings of the one or more audio-visual devices on the basis of data acquired from the AV controller.

4. The endoscopic system according to claim 1, further comprising:
a first recording medium for storing data for use in activating the one or more audio-visual devices, which is accessible by the system controller;
an input device for entering information for retrieving the data from the first recording medium; and
a second recording medium for storing data for use in activating the one or more audio-visual devices, which is accessible by the AV controller;
wherein the system controller has a function of transmitting the data retrieved from the first recording medium according to the information entered from the input device to the AV controller and the AV controller has a function of setting the corresponding data recorded in the second recording medium according to the data transmitted from the system controller.

5. The endoscopic system according to claim 1, further comprising a recording medium for storing data for use in activating the one or more audio-visual devices, which is accessible by the AV controller,
wherein the system controller has a function of establishing a link with the AV controller and setting the data stored in the recording medium via the input device of the system controller.

6. In an endoscopic system having a controller for controlling at least an endoscope and an AV controller for controlling one or more audio-visual devices, a method of controlling the one or more audio-visual devices from the controller comprising:
transmitting data for use in activating the one or more audio-visual devices from the controller to the AV controller; and
setting operating conditions corresponding to the data.
